(19) United States
(12) Patent Application Publication
(10) Pub. No.: US 2013/0102788 A1
(43) Pub. Date: Apr. 25, 2013

(54) Method for Synthesizing 1,3,5-Tri-(N-Phenylbenzimidazolyl) Benzene

(71) Applicants: Lifei Cai, Beijing (CN); Lei Dai, Beijing (CN); Hongyu Zhao, Beijing (CN); Yanfeng Ding, Beijing (CN); Weilong Zhang, Beijing (CN); Junhao Lian, Beijing (CN)

(72) Inventors: Lifei Cai, Beijing (CN); Lei Dai, Beijing (CN); Hongyu Zhao, Beijing (CN); Yanfeng Ding, Beijing (CN); Weilong Zhang, Beijing (CN); Junhao Lian, Beijing (CN)

(73) Assignees: BEIJING AGLAIA TECHNOLOGY DEVELOPMENT CO., LTD., Beijing (CN); GUANGDONG AGLAIA OPTOECTRONIC MATERIALS CO., LTD., Foshan (CN)

(21) Appl. No.: 13/582,060

(22) PCT Filed: Feb. 9, 2011
(86) PCT No.: PCT/CN2011/070889

§ 371 Date: Aug. 31, 2012

(30) Foreign Application Priority Data
Jul. 12, 2010 (CN) 201010223992.6

(51) Int. Cl. C07D 403/14

(52) U.S. Cl. CPC C07D 403/14
USPC 548/305.4

(57) ABSTRACT

The present invention relates to efficient synthetic method of 1,3,5-tris (N-Phenylbenzimidazole) Benzene and pertains to the field of synthesis of organic light-emitting materials. The method and process of the patent are as following: 1,3,5-tris (N-Phenyl-N Phenylammonium) Benzamide is treated by shrinking agent but no high temperature required, and then high purity product can be reached after raw material dissolved. The reaction only takes a few minutes and through recrystallization the purity can be over 99%, and then the material with stable crystal phase reached after sublimation step. The method introduced by the present patent is easy to operate, and with high in yield and purity, which adapt industrial production and has important significance for the industrialization.

### THE TECHNICAL FIELD BELONGS

The invention belongs to the field of synthesis of organic light-emitting materials, especially which involving the rapid and efficient synthetic methods for 1,3,5-tris (N-Phenylbenzimidazole) Benzene, and the improvement of purification.

### TECHNICAL BACKGROUND

In 1987, Ching W. Tang, et al from Kodak Company successfully prepared sandwich-type bilayer organic light-emitting diodes by using Alq₃ as light-emitting layer and Aromatic Di-Amine as hole-transporting [A1] layer. (Tang C. W., et al. Applied Physics Letters, 1987, 51, 913). In 1990, Burroughes J. H., et al from Cambridge University in England built light-emitting devices (LEDs) based on organic polymer (Burroughes J. H., et al. Nature, 1990, 347, 5395).

These breakthroughs greatly promoted the development of organic light-emitting technical field. Since then, scientists in different countries devoted much energy to the research and development in this field. More and more organic light-emitting materials were developed and applied. Out of all kinds of organic light-emitting materials, 1,3,5-tris (N-Phenylbenzimidazole) Benzene, which is a kind of widely used electron transport material, because of its large size of HOMO, LUMO, and triplet energy level, has been used as hole-blocking material and phosphorescence host material.

1,3,5-tris (N-Phenylbenzimidazole) Benzene, which reported on the patent U.S. Pat. No. 5,645,948 of Kodak, is only fit for the application testing in laboratory but not for the industrialization. The synthetic method reported on the patent U.S. Pat. No. 5,645,948, which mainly through pressure-relief and high temperature reached ring closure with 20% of yield rate, and along with twice the column chromatography and sublimation the production can be used followed the purification process, which is far away the requirement of the industrialization. Enlightened by the patent, N-Phenyl-Imidazolyl was introduced into different molecules, and bipolar molecule reached which can be used as phosphorescence host material and material. (ChuLo Yang et al, j. phys. Chem. C, 2010, 114, 5193; Ziyi Ge, Teruaki Hayakawa, et al. Adv. Funct. Mater. 2008, 18, 584-590; Yuan-Li Liao, Chi Yen Lin, Ken-Tsung Wong, Tei-Hung Hou, and Wen-Yi Hung, org_lett, 07, 9, 4511). And reached materials can be used as dual-functional material as well as phosphorescence host material. However, the electron-transporting material is still impossible that replaced by 1,3,5-tris (N-Phenylbenzimidazole) Benzene. The lack of synthetic methods, which means hardly meet the demand for the industrialization, limit the application of the material. The research on 1,3,5-tris (N-Phenylbenzimidazole) Benzene is only restricted in using in devices.

### THE CONTENTS OF THIS INVENTION

In view of these shortcomings mentioned above, we present in this invent an efficient method of which the synthesis of 1,3,5-tris (N-Phenylbenzimidazole) Benzene turns to be easily operated and adapts industrial production with high yield rate, which has important significance for the industrialization.

The efficient synthetic method of 1,3,5-tris (N-Phenylbenzimidazole) Benzene in formula (I) includes as following the major steps: (1) 1,3,5-(N-Phenyl-N-Phenyl aniline) Benzamide was prepared and its molecular structure see formula (II), (2) Treated by shrinking agent and heated at 80-200° C., reached compound see formula (I).

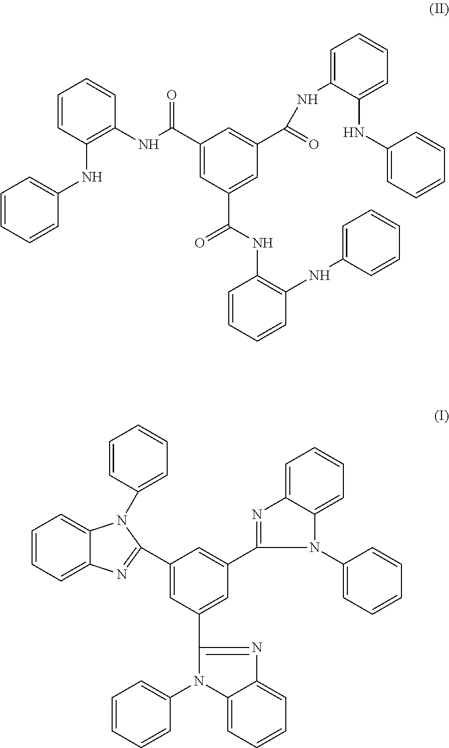 [SMILES: O=C(Nc1ccccc1Nc1ccccc1)c1cc(C(=O)Nc2ccccc2Nc2ccccc2)cc(C(=O)Nc2ccccc2Nc2ccccc2)c1 ; c1ccc(-n2c(-c3cc(-c4nc5ccccc5n4-c4ccccc4)cc(-c4nc5ccccc5n4-c4ccccc4)c3)nc3ccccc32)cc1]

The shrinking agent we used is Phosphoric acid, poly Phosphoric acid, concentrated sulfuric acid, the Acetic acid, the mixture of Acetic acid and Hydrochloric acid, Phosphorus Trichloride, and Phosphorus Oxychloride.

The preferable reaction temperature is at 80-180° C.

And optimum reaction temperature 80-160° C.

The synthetic method mentioned above also includes the purification step which through the distillation method.

The distillation temperature is 170-380° C., and the deposition temperature 25-150° C.

The preferable distillation temperature is 190-360° C., and the deposition temperature 25-130° C.

And optimum distillation temperature is 190-340° C., the deposition temperature 25-100° C.

The synthetic method in the presented patent improved based on the patent U.S. Pat. No. 5,645,948 published by Kodak Company. During the step ring closure, three water molecules were removed through condensation method, and with shrinking agent adopted which allowed that the reaction conditions can be implemented at lower temperature and shortened the reaction process to a few minutes, higher in yield. The presented synthetic method is easy to implement and to apply in industry, and consequently so important to apply the compound formulated in formula (I) in industry.

1,3,5-(N-Phenyl-N-Phenyl aniline) Benzamide is a polycrystalline compound which easily crystallizes when in use, consequently with the life of devices shortened. Crystallization can be avoided by post treatment, and the produced material with structural stability can be adopted directly. In experimenting we find out that the products synthesized using the method on American patent at high temperature, can avoid crystallization and with unchangeable properties even then dissolved in a solvent. And we also reached the same consequence by distillation. By comparing four sample treatments for DSC in order to research the application of the material in devices, we found that with different cooling routing, the produced material would be different in structure. If shrink reaction temperature we adopted is lower than recrystallization temperature, then polycrystalline materials reached. And if purification temperature higher than recrystallization temperature, then single crystal material reached. Furthermore, the processes we adopted only take a few minutes and adapt industrialization. Recrystallization can be adopted to increase the purity but unfeasible for the problem of polycrystalline. The unstable crystals can be removed by annealing treatment or distillation the purification step, and the proper materials reached. Annealing treatment is suitable mostly for the manufacture processes or the preparation of small samples. By adjusting the conditions of distillation and sedimentation, the high purity material can be reached with stable crystal phase.

The advantages of the preparation process of the present invention are as following: The synthesis of 1,3,5-(N-Phenyl-N-Phenyl aniline) Benzamide, with no need of high temperature, the first step was finished by either adopting the provided solvent by the present patent or other methods. In the first step reaction, when intermediate dissolved the high purity product reached, which lasted only a few minutes. After recrystallized, the purity can be over 99%. Then under the condition of distillation, the stable crystal phase reached.

### DESCRIPTION OF FIGURES

FIG. 1 shows the HNMR spectra of embodiment 1.

FIG. 2 shows the first DSC result of the product of embodiment 1.

FIG. 3 shows that, after the first quenched, the second DSC result of the product of embodiment 1.

FIG. 4 shows the cooling chart after sublimated of the product of embodiment 1.

FIG. 5 shows the cooling chart with no sublimating of the product of embodiment 1.

FIG. 6 shows the first DSC result of the control group product.

FIG. 7 shows that, after the first quenched, the second DSC result of the control group product, and recrystallization occurred at 196.92° C.

### THE DETAILED METHOD AND BASIC PROCESSES

Some details will have to be further elaborated as we go along with patent embodiments.

### Embodiment 1

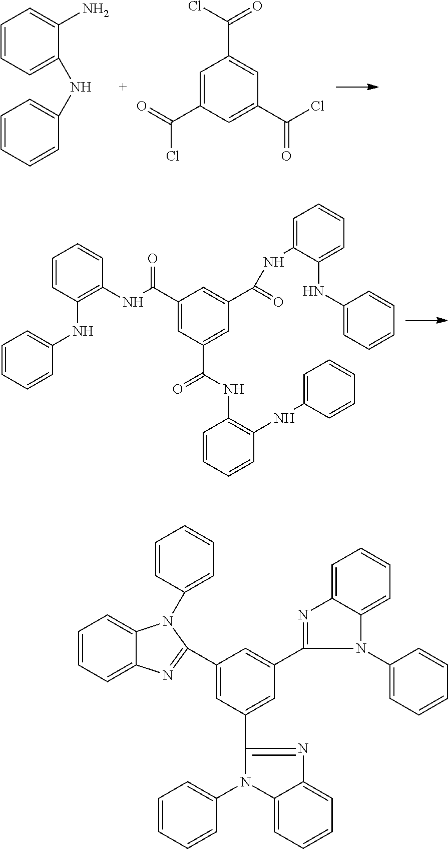 [SMILES: Nc1ccccc1Nc1ccccc1 ; O=C(Cl)c1cc(C(=O)Cl)cc(C(=O)Cl)c1 ; O=C(Nc1ccccc1Nc1ccccc1)c1cc(C(=O)Nc2ccccc2Nc2ccccc2)cc(C(=O)Nc2ccccc2Nc2ccccc2)c1 ; c1ccc(-n2c(-c3cc(-c4nc5ccccc5n4-c4ccccc4)cc(-c4nc5ccccc5n4-c4ccccc4)c3)nc3ccccc32)cc1]

### Step One

Dissolved 289.00 G of N-Phenyl-o-Phenylenediamine in 1 L of THF and reached bronzing solution. Then dissolved in 126.10 G of trimesoyl Chloride at room temperature, the solution turned to be brownish black and with no change in the temperature. Then the solution after stable overnight were poured into 3 L of water, a great mass of oily matter occurred. After cooled and lumped, filtered and dried, 450 G of brown solid reached, with 98% of yield rate.

### Step Two

450 G of products reached by step one was mixed with 2 L of Phosphoric acid, and bronzing turbid solution reached. Then warmed up and with the increase of temperature, the redissolution of the precipitates finally finished in 2 minutes after the temperature reached 100° C. Then detected with TLC, no raw material was found. Then after cooling and poured into water, a great mass of light pink solid matters were separated out. By stoving and recrystallized by using Dichloromethane, 400 G of products reached. The hydrogen spectrums see FIG. 1.

### Control Group

1,3,5-tris (N-Phenylbenzimidazole) Benzene was synthesized according to the method reported on the patent U.S. Pat. No. 5,645,948 of Kodak.

### Experimental Group

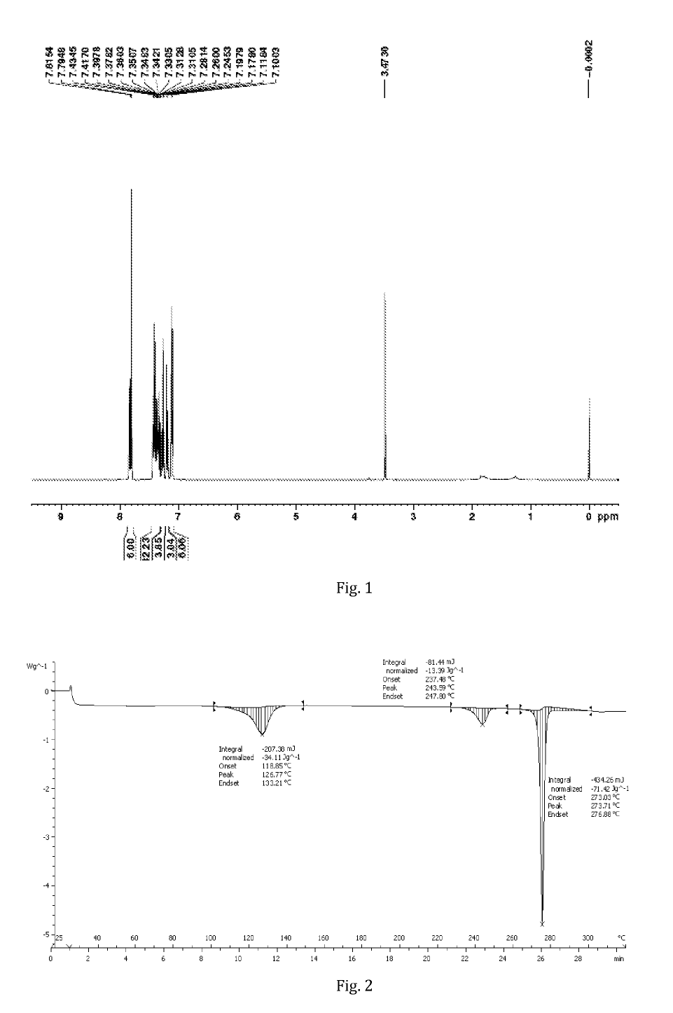

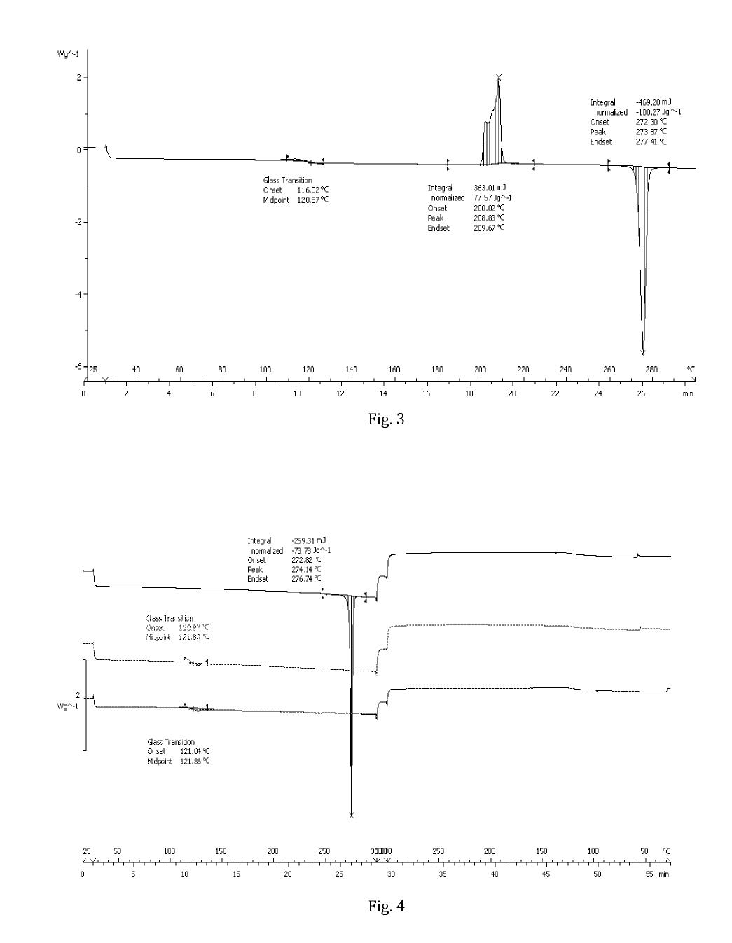

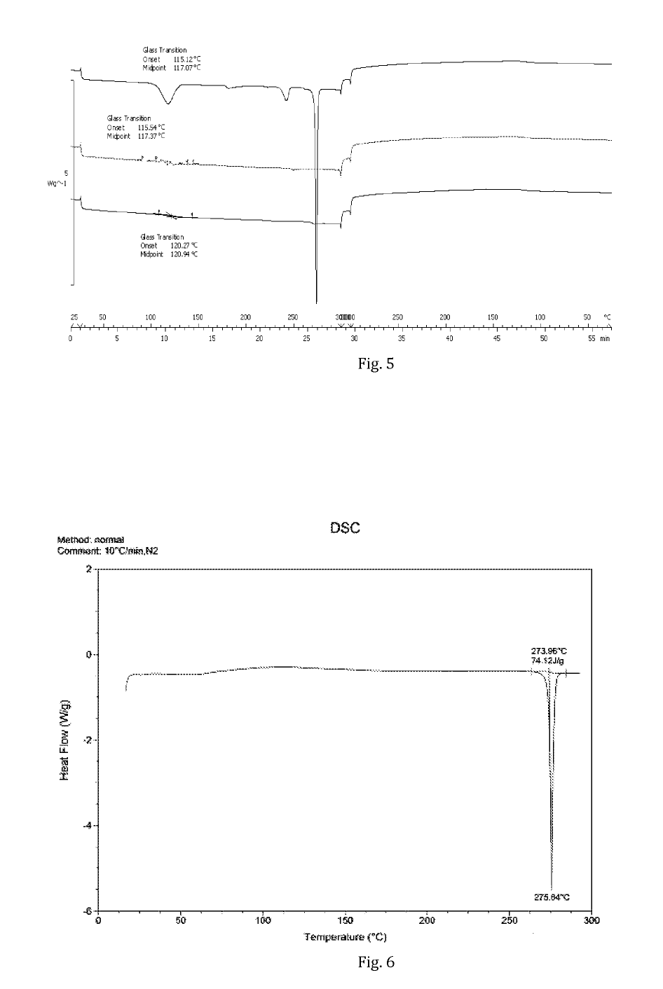

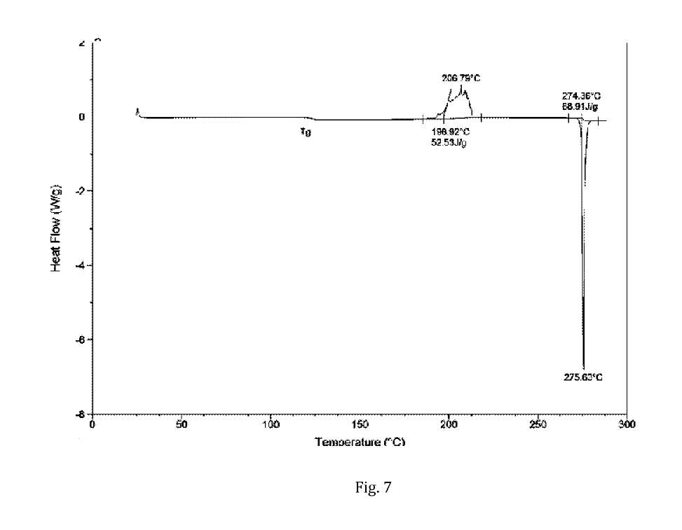

(1) The first DSC graphs of the products by the method of the present patent with no sublimation step see FIG. 2. We can see 126.77° C. and 243.99° C. are the points for the crystal change.
(2) The products by the method of the present patent with no sublimation step, heated to 300° C. and then quenched to 25° C., and then heated the second time, the graphs see FIG. 3. The interval 200-209° C. is for the crystalline peak.
(3) The products by the method of the present patent with sublimation step, detected by DSC three times, the interval 30-55 minutes is for cooling graph see FIG. 4, with the cooling by 10° C./min. And even heated for the second time, no crystal occurred with stable amorphous state.
(4) The products by the method of the present patent with no sublimation step, detected by DSC three times, the interval 30-55 minutes is for cooling graph see FIG. 5, with the cooling by 10° C./min. And even heated for the second time, no crystal occurred with stable amorphous state.
(5) The first DSC graphs of the control group products with no sublimation step see FIG. 6.
(6) The control group products with no sublimation step, heated to 300° C. and then quenched to 25° C., and then heated the second time, the graphs see FIG. 3. We can see that crystals occurred at 196.92° C.

Conclusions: The method and process we introduced in the patent are simple and adapt industrialization. However, the reached products are unstable crystals. After sublimation step and the unstable crystals removed, the end-products are as same as which synthesized by using the method of Kodak's patent. But Kodak's method brings the low yield rate which means unfeasible for industrialization. Detected the properties of materials by DSC, we knew that the material after heated, melted, and cooled in order, turned to be amorphous, but if quenched by liquid nitrogen, then crystal reached. And the crystallizing point is 196° C., which means the material can be used for OLED devices. Therefore, we say that the efficient synthetic methods introduced in the present patent have given a basis for the fabrication of OLED devices.

1. A method for preparing 1,3,5-tris (N-phenylbenzimidazole) benzene of formula (I), comprising the steps of:
(1) preparing 1,3,5-(N-phenyl-N-phenyl aniline) benzamide of formula (II); and
(2) treating the 1,3,5-(N-phenyl-N-phenyl aniline) benzamide with a shrinking agent and heating at 80-200° C. to obtain 1,3,5-tris (N-phenylbenzimidazole) benzene.

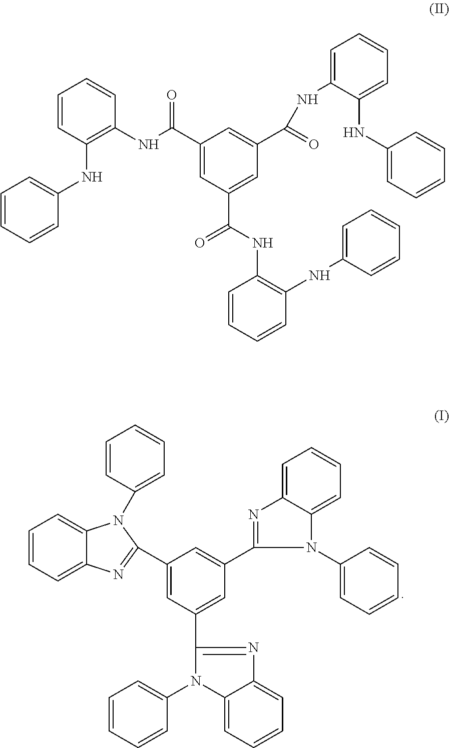 [SMILES: O=C(Nc1ccccc1Nc1ccccc1)c1cc(C(=O)Nc2ccccc2Nc2ccccc2)cc(C(=O)Nc2ccccc2Nc2ccccc2)c1 ; c1ccc(-n2c(-c3cc(-c4nc5ccccc5n4-c4ccccc4)cc(-c4nc5ccccc5n4-c4ccccc4)c3)nc3ccccc32)cc1]

2. The method of claim 1, wherein the shrinking agent is selected from the group consisting of phosphoric acid, poly(phosphoric) acid, concentrated sulfuric acid, acetic acid, and a mixture of acetic acid and hydrochloric acid, phosphorus trichloride and phosphorus oxychloride.

3. The method of claim 1, wherein the heating is at 80-180° C.

4. The method of claim 3, wherein the heating is at 80-160° C.

5. The method of claim 1, further comprising a step of purifying the 1,3,5-tris (N-phenylbenzimidazole) benzene by distillation.

6. The method of claim 5, wherein the distillation temperature is 170-380° C.

7. to the method of claim 6, wherein the distillation temperature is 190-360° C.

8. The method of claim 7, wherein the distillation temperature is 190-340° C.

9. The method of claim 1, further comprising a step of purifying the 1,3,5-tris (N-phenylbenzimidazole) benzene by deposition.

10. The method of claim 9, wherein the deposition temperature is 25-150° C.

11. The method of claim 10, wherein the deposition temperature is 25-130° C.

12. The method of claim 11, wherein the deposition temperature is 25-100° C.
